(19) United States
(12) Patent Application Publication
(10) Pub. No.: US 2012/0095437 A1
(43) Pub. Date: Apr. 19, 2012

(54) AUTOMATIC CONTROL SYSTEM AND METHOD FOR THE CONTROL OF ANESTHESIA

(76) Inventor: Thomas Hemmerling, Montreal (CA)

(21) Appl. No.: 13/124,778

(22) PCT Filed: Oct. 19, 2009
(86) PCT No.: PCT/CA09/01491

§ 371 Date: Jan. 3, 2012

Related U.S. Application Data
(60) Provisional application No. 61/136,960, filed on Oct. 17, 2008.

(51) Int. Cl. A61M 5/168

(52) U.S. Cl. 604/503; 604/65; 604/66; 604/500

(57) ABSTRACT

The present invention relates to an automatic anesthesia control system and method for the detection of the degree of each component of general anesthesia, automatic detection of drugs administered and automatic drug delivery via intravenous route.

### TECHNICAL FIELD

The present invention relates to an automatic control system and method for the control of anesthesia.

### BACKGROUND

Anesthesia defines a state where a patient is unconscious (‘anesthesia’, ‘hypnosis’ are similarly used terms to describe this state), pain free (‘analgesia’, ‘nociception’ are similarly used terms to describe this state) and his or her muscles are relaxed (‘neuromuscular blockade’, ‘neuromuscular block’ are similarly used terms to describe this state). All three components are in various degrees necessary to provide what is called general anesthesia; partial forms of general anesthesia are sedation, where the patient is in a degree of unconsciousness, sleepy state, analgesia where only pain free state is achieved and maintained to allow certain procedures or interventions. For all three components of general anesthesia, certain parameters are used to determine the varied degree of each component.

### Hypnosis

In order to determine hypnosis, parameters are based on either spontaneous electroencephalogram-derived parameters or evoked potentials, which can be auditory or any other form of sensory potentials and are created after stimulation.

### Analgesia

In order to determine analgesia, surrogates are used whenever communication with the patient is impossible; many parameters can be used, hemodynamic parameters, such as heart rate or blood pressure or derivates of these, body reactions to pain, such as sweating, changes in lacrimation, pupil size, changes of hand conductance, reaction to deliberately evoked pain stimuli unrelated to surgery or the intervention or other forms of body reactions to pain.

### Muscle Relaxation

In order to determine muscle relaxation, the muscle force can either be determined directly, depending on the ability of the patient to obey commands, or indirectly by stimulation of a motor nerve and determining the muscle contraction by various means, most commonly by directly measuring the force, the acceleration of contraction, the electric equivalent of muscle contraction, the movement as such or the sounds created by muscle relaxation.

Anesthesiologists use the variables of these parameters for each component and estimate the drug dose which has to be administered. This can be achieved by administering one to three or more drugs into the body of the patient by intravenous, inhalational, intramuscular or subcutaneous way. One of the most common forms of anesthesia drug delivery is the intravenous route; this can be achieved by either continuous intravenous or intermittent intravenous administration. Drugs for anesthesia are usually delivered in vials and have to be transferred into syringes of different size in order to be administered by intravenous route. Labeling of these vials is done generally manually.

### SUMMARY

The present invention relates to an anesthesia control system for controlling at least one component of anesthesia of a patient, comprising:
an input for acquiring a target parameter indicative of a current degree of the at least one component of anesthesia of the patient from a monitoring module;
an output for connecting to a drug delivery mechanism being so configured so as to be intravenously infuse a drug to the patient;
a user interface for activating and controlling the control system;
a processor operatively connected to the input, the output and the user interface, the processor being so configured so as to:
   calculate the necessary dose of a drug for achieving a desired degree of the at least one component of anesthesia based on the target parameter; and
   providing the calculated drug dose to the output.

The present invention further relates to a method for controlling at least one component of anesthesia of a patient, comprising the step of:
acquiring a target parameter indicative of a current degree of the at least one component of anesthesia;
calculating the necessary dose of a drug for achieving a desired degree of the at least one component of anesthesia based on the target parameter; and
delivering the drug to the patient via an infusion drip.

### BRIEF DESCRIPTION OF THE FIGURES

Embodiments of the invention will be described by way of example only with reference to the accompanying drawings, in which:

FIG. 1 is a schematic view of the automatic anesthesia control system in use in accordance with an illustrative embodiment of the present invention;

FIG. 2a is a schematic view of the back of the of the automatic anesthesia control system of FIG. 1 in accordance with an illustrative embodiment of the present invention;

FIG. 2b is a schematic view of the back of the of the automatic anesthesia control system of FIG. 1 in accordance with a second illustrative embodiment of the present invention;

FIG. 2c is a schematic view of the back of the of the automatic anesthesia control system of FIG. 1 in accordance with a third illustrative embodiment of the present invention;

FIG. 3 is a block diagram of the control components of the automatic anesthesia control system of FIG. 1;

FIG. 4 is an example of a startup screen from a graphical interface for use with the automatic anesthesia control system of FIG. 1;

FIG. 5 is an example of a maintenance screen from a graphical interface for use with the automatic anesthesia control system of FIG. 1;

FIG. 6A and 6B are a flow diagram of an illustrative example of the hypnosis monitoring process based on the bispectral index (BIS);

FIG. 7 is an example of a user sedation screen from a graphical interface for use with the automatic anesthesia control system of FIG. 1; and

FIG. 8 is an example of a low saturation warning pop-alert from a graphical interface for use with the automatic anesthesia control system of FIG. 1.

### DETAILED DESCRIPTION

Generally stated, the non-limitative illustrative embodiment of the present invention provides an automatic control system and method for the control of anesthesia, automatic detection of some or all components of general anesthesia, automatic drug recognition, and automatic drug delivery. As the automatic anesthesia control system operates in a closed-loop configuration, it continuously samples control variables and changes the drug delivery at a more rapid rate than an anesthetist would with a manual delivery system, which results in a greater stability of the control variables.

General anesthesia consists of three different components: hypnosis, analgesia and muscle relaxation. For each component, one target parameter is identified reflecting the actual status of this component, meaning one parameter reflecting the current degree of hypnosis, one parameter reflecting the current degree of analgesia and one parameter reflecting the current degree of muscle relaxation. Once these parameters are identified, they serve as control variables used by the automatic anesthesia control system. It is to be understood that in an alternative embodiment multiple target parameters may be used for one or more of the anesthesia components.

The desired degree for each component is achieved by administering a different drug (i.e. three different drugs) via infusion syringe pumps. The infusion rates are based on algorithms built into the automatic anesthesia control system. For example, in an illustrative embodiment of the present invention, the following parameters are used: bispectral index (BIS) for the hypnosis component, analgoscore for the analgesia component and phonomyography for the muscle relaxation component. The automatic anesthesia control system connects with standard vital sign monitors in order to calculate set targets which are maintained through feedback control. It is to be understood that other parameters may be used, for example any parameter indicating a degree of unconsciousness for the hypnosis component and a parameter indicating a degree of pain for the analgesia component.

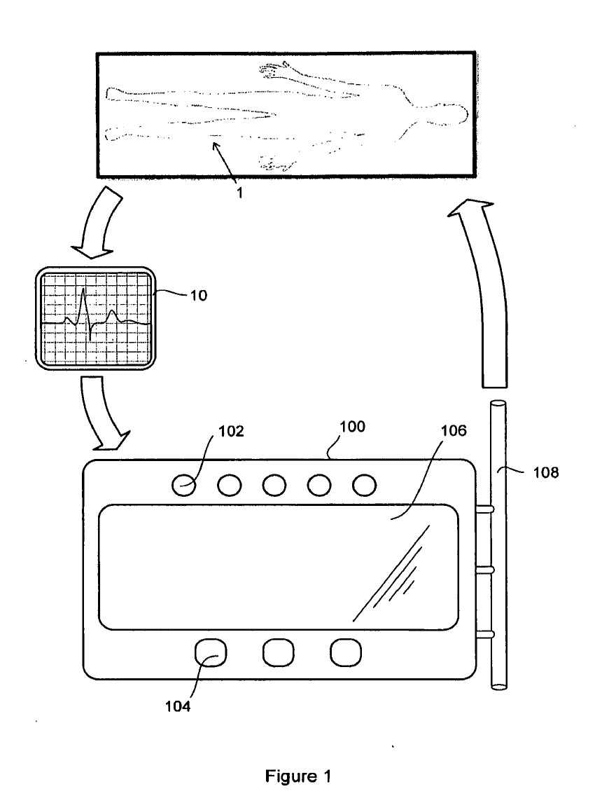

Referring to FIG. 1, there is shown the schematic diagram of the use of the automatic anesthesia control system 100, which generally consists of control buttons 102, 104, a display 106, with or without a touch-screen surface. The control buttons 102, 104 can be used by a user to interact with the automatic anesthesia control system 100; possible interactions are, for example, choice between fully automatic or semi-automatic operating mode, data entering, trend retrieval, change of display mode or any other human-machine interaction. The automatic anesthesia control system 100 may communicate directly (e.g. by cable, etc.) or indirectly (e.g. by wireless, etc.) with other systems, or retrieve variables from the body of the patient 1. These data are used as input into the automatic anesthesia control system 100 to reflect the patient's 1 state of anesthesia.

The body of the patient 1 delivers several variables which can be monitored by a monitoring module 10 and used to record the target parameters, giving an objective status of the three components of general anesthesia. These variables are acquired by the monitoring module 10 either automatically via direct or indirect data transfer. The monitoring module 10 acts as a filtering module (filtering distinction can be varied) and delivers all necessary data to the automatic anesthesia control system 100. This can be performed via direct or indirect data transfer. The automatic anesthesia control system 100 uses built-in algorithms in order to record, display and analyze the data it receives. Manual input by a user is possible via sets of control buttons 102, 104 or, optionally, through a touch-display 106 or keyboard. The algorithms will be further detailed below. An example of a monitoring module 10 that may be used if the Lifegard II™ non-invasive vital signs monitor from CASMED Inc. It is to be understood that other monitoring equipment may be used and that in an alternative embodiment the monitoring module 10 may be implemented within the automatic anesthesia control system 100 itself.

Depending on which mode of operation the automatic anesthesia control system 100 is operated, one or more components of general anesthesia can be automatically controlled; manual administration of any component is also possible at any given moment of time via the control buttons 102, 104. Using automatic or manual mode, the drugs are then delivered directly or indirectly into the human body 1 via an infusion drip 108.

The automatic anesthesia control system 100 calculates the necessary doses of any drug used to perform anesthesia; drug administration can then be performed directly via built-in syringe drives loaded with syringes, via a built-in syringe drive with a drug cartridge system or indirectly via communication directly or indirectly with standard infusion pumps.

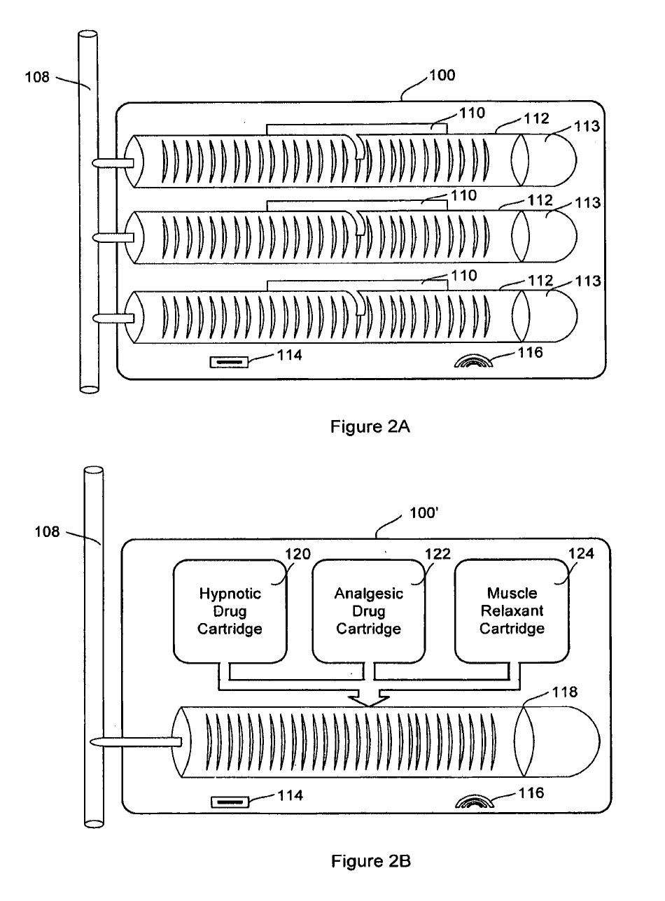

Referring to FIG. 2a, there is shown a schematic view of the back of a first embodiment of the automatic anesthesia control system 100 having a drug delivery mechanism composed of three syringe holders 110, having hook-like mechanism or any other attachment mechanism, with associated syringe drives 113 are disposed for receiving any standard syringe 112. The syringes 112 are then connected to the body of the patient 1 via the infusion tubing 108. Possible communication ports may be integrated for direct 114 or indirect 116 communications with other systems. It is to be understood that in a further alternative embodiment, the number of syringe holders 110 and associated syringe drives 113 may vary.

Referring to FIG. 2b, there is shown a schematic view of the back of a second embodiment of the automatic anesthesia control system 100′ having a drug delivery mechanism composed of three component drug cartridges: an hypnotic drug cartridge 122, an analgesic drug cartridge 124 and a muscle relaxant cartridge 126 are connected to a direct syringe drive 118 for automatic delivery of the drugs. The direct syringe drive 118 is then connected to the body of the patient 1 via the infusion tubing 108. User interaction consists of inserting the different drug cartridges 122, 124, 126 into their associated cartridge holders (not shown). False drug application is prevented using automatic drug recognition based on, for example, bar code recognition of the drug cartridges. Possible communication ports may be integrated for direct 114 or indirect 116 communications with other systems. It is to be understood that in a further alternative embodiment, the number of drug cartridges 122, 124, 126 and associated cartridge holders may vary.

Referring to FIG. 2c, there is shown a schematic view of the back of a third embodiment of the automatic anesthesia control system 100″; in this embodiment no syringe holders and syringe drive or direct syringe drive are available. The automatic anesthesia control system 100″ communicates directly, via direct communication port 114, or indirectly, via indirect communication port 116, with standard infusion pumps and drug delivery is then performed using these pumps.

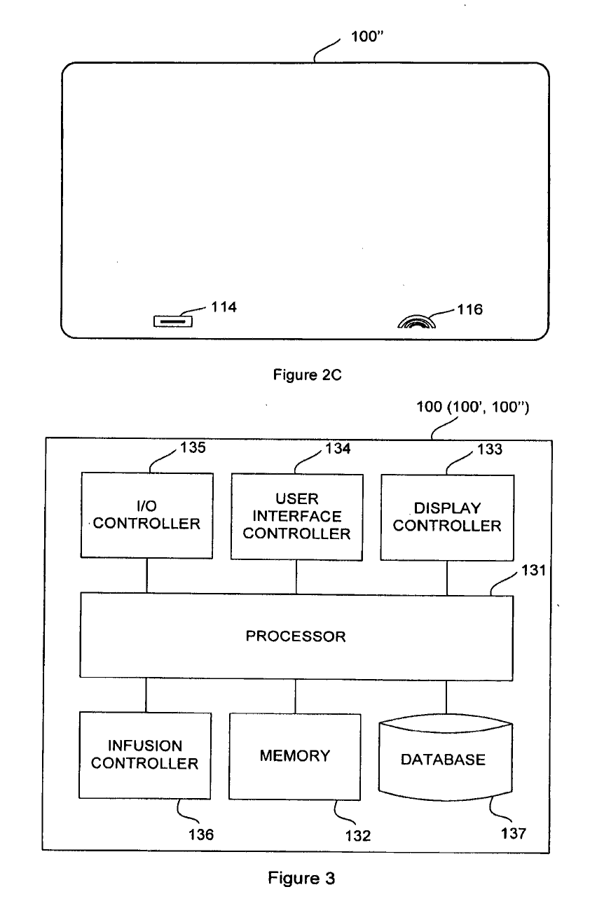

Referring now to FIG. 3, there is shown a block diagram of the control components of the automatic anesthesia control system 100 (100′, 100″), which generally comprise a processor 131 with an associated memory 132, a display controller 133, a user interface controller 134, an input/output (I/O) controller 135, an infusion controller 136 and a database 137. The memory 132 contains the various algorithms executed by the processor 131 to provide the functionalities of the automatic anesthesia control system 100. Interaction with the user is provided by the display controller 133 which displays the various graphical user interfaces via the screen 106 while the user interface controller 134 controls data and control input from the control buttons 102 and 104, optional touch-screen 106. In an alternative embodiment, a keyboard, mouse or other such data entry means may be handled by the user interface controller 134 to input data and commands. An input/output (I/O) controller controls the possible communication ports for direct 114 or indirect 116 communications with other systems. An infusion controller 136 controls the actuation of the syringe drives 113, the drug cartridges 122, 124, 126 and the direct syringe drive 118. Finally, a database 137 contains different types of surgeries which associated patterns of anesthesia and patient data.

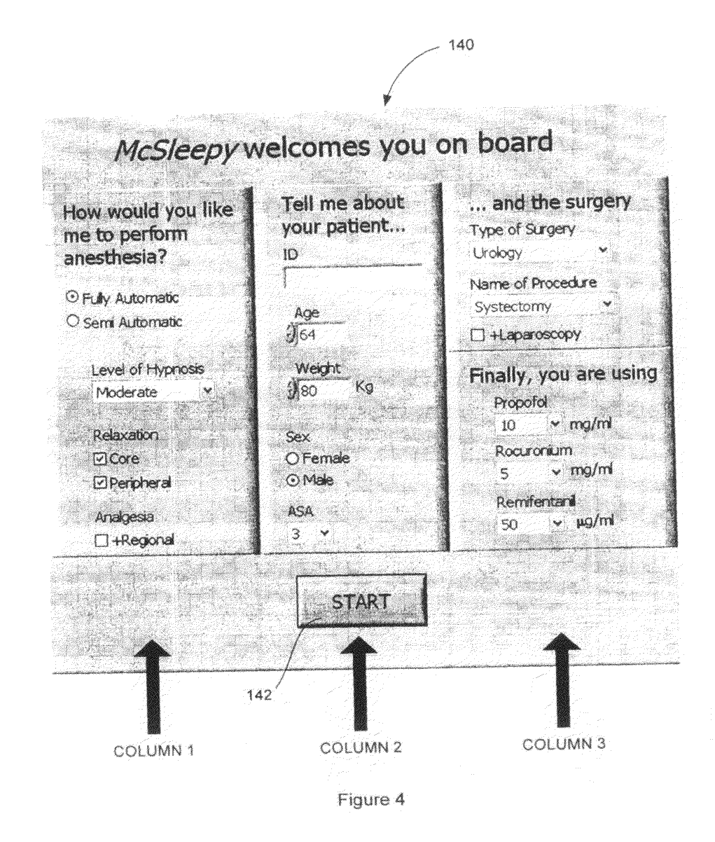

Referring to FIG. 4, there is shown an example of a startup screen 140 from a graphical interface for use with the automatic anesthesia control system 100. The screen 140, which is displayed in display 106, is divided in three columns: column 1, column 2 and column 3.

In the left column, column 1, the user may choose between a fully-automatic mode and a semi-automatic mode. In the automatic mode all three components of anesthesia, i.e. hypnosis, analgesia and muscle relaxation, are controlled fully automatically by the automatic anesthesia control system 100, which chooses appropriate targets for level of hypnosis, analgesia and the type/level of muscle relaxation, i.e. core relaxation (relaxation of profound muscles) or peripheral relaxation (relaxation of extremity muscles), according to the type of procedure performed.

In the semi-automatic mode the user can choose which components of anesthesia are to be automatically administered by the automatic anesthesia control system 100. For example, the user may choose the level of hypnosis he or she would like, the level of analgesia (depending on co-analgesic modes such as regional anesthesia) and the type/level of muscle relaxation, i.e. core relaxation (relaxation of profound muscles) or peripheral relaxation (relaxation of extremity muscles). The monitoring and manual administration of anesthetic drugs is performed by the user using the control buttons 102, 104 and display 106 whilst the automatic anesthesia control system 100 automatically administers the remaining drug(s), continuously informing the user about its status of delivery and effect in real time.

In the middle column, column 2, the user may enter relevant patient information, such as an ID number, age, weight, sex or classification of the severity of the patient's disease, in this example with an ASA classification. It is to be understood that additional information may be included.

Finally, in the right column, column 3, the user may enter surgery-relevant information such as type of surgery, exact description of surgery (for example with or without laparoscopy) as well as the concentration and names of the drugs he or she would like to use. It should be noted, however, that in the alternative embodiment of the automatic anesthesia control system 100′ shown in FIG. 2B, the names of the drugs may be automatically filled by the automatic drug recognition feature. Alternatively, the user may enter the name of the drug which will be used to validate the drug cartridges inserted in the automatic anesthesia control system 100′.

When the user is done entering the various information, he or she then presses the ‘start’ button 142 to initialize the automatic anesthesia control system 100′ after which, automatically, the maintenance screen appears.

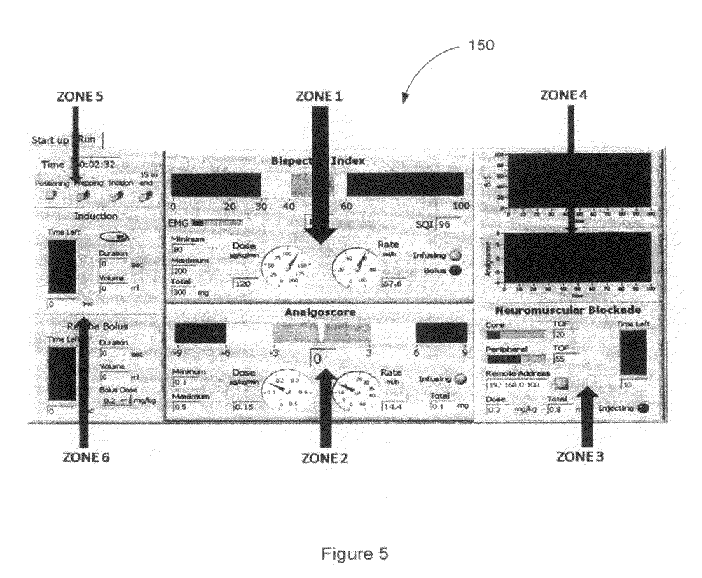

Referring now to FIG. 5, there is shown an example of a maintenance screen from a graphical interface for use with the automatic anesthesia control system 100. The maintenance screen 150, which is displayed in display 106, is separated in different zones, zone 1 to zone 6, according to their importance (i.e. zone 1 is more important than zone 6), which is reflected in their placement (e.g. middle, up) and size (e.g. bigger) on the maintenance screen 150.

Each zone displays information as follows:
zone 1 monitors hypnosis and the infusion rate of the hypnotic agent;
zone 2 monitors analgesia and the infusion rate of the analgesic agent;
zone 3 monitors core and peripheral relaxation, and the infusion rate of the muscle relaxation agent;
zone 4 graphically shows trends for hypnosis and analgesia;
zone 5 provides information about different time points of surgery and elapsed time of operation of the automatic anesthesia control system 100; and
zone 6 provides a time indicator for induction and boli.

More specifically, in zone 1 there is displayed in realtime the level of hypnosis, which may be provided, for example, using the BIS from Aspect Medical Systems, the infusion dose (actual infusion rate) of the drug (for example propofol) in two different units (μg/kg/min or ml/h) as well as the status of the infusion (on or off).

In zone 2, there is displayed in realtime parameters concerning the analgesia, infusion dose (actual infusion rate) of the drug (for example remifentanil) as well as a parameter for the level of pain, for example the analgoscore as disclosed in PCT Patent Application publication No. WO 2008/086624.

In zone 3, there is displayed in realtime parameters concerning the muscle relaxant, infusion dose (actual infusion rate) of the drug (for example rocuronium) and relaxation of, for example, the corrugator supercilii (CS) and adductor pollicis (AP) muscles, indicating core and peripheral muscle relaxation.

As for zone 5, surgery related time points may be provided to the automatic anesthesia control system 100, namely positioning, prepping of the patient, incision and time to the end of surgery, which are important time factors that allow it to adjust the doses of the drug infusions. For example, most anesthesiologists would increase the infusion rate of remifentanil 5 mins prior to the incision to ‘anticipate’ the increased pain stimulus due to the surgical incision. Accordingly, the automatic anesthesia control system 100 will automatically increase the dose of remifentanil at the time point ‘prepping’.

It is to be understood that the above-mentioned screens are mere examples of possible user interfaces.

### Algorithms for Use in the Fully Automatic Mode

As mentioned previously, the database 137 is populated with different types of surgeries which are grouped according to different ‘patterns’ of anesthesia (level of hypnosis, level of pain stimulus, type and degree of muscle relaxation). These patterns of anesthesia are then used by control algorithms residing in the memory 132 and executed by the processor 131.

For example, lets assume the selected surgery was an endoscopic gallbladder resection. This kind of surgery is characterized by a short duration (ca. 1h), high level of pain stimulus during, but not after surgery, necessity of profound muscle relaxation (for a relatively short period) and is mostly done on an outpatient level (i.e. the patient should be able to leave the hospital on the same day in order to limit financial and human resources for this type surgery.

This clinical scenario translates into the following categories (one category for each component of general anesthesia):
hypnosis level: light;
analgesia level: profound;
muscle relaxation level: profound; and site of muscle relaxation: core.

Light hypnosis guarantees early recuperation after general anesthesia, profound analgesia guarantees best stress suppression and profound core muscle relaxation guarantees optimal surgical conditions at the abdominal cavity.

In addition, short acting drugs may be suggested by the automatic anesthesia control system 100 in this case since pain after surgery is light (therefore no need for intraoperative use of an analgesic drug which has a prolonged action after surgery), and recovery from surgery should be swift in order to facilitate early discharge from the hospital. In this example, the automatic anesthesia control system 100 may propose the combination of propofol, remifentanil and rocuronium.

Pattern learning

In an alternative embodiment, the automatic anesthesia control system 100 may ‘learn’ from manual adjustments made by a specific user for a given type of surgery and store the ‘learnt’ time patterns in its database 137 for future reference. For example, after a given number of performed cases of anesthesia for endoscopic gallbladder resection with user X, the automatic anesthesia control system 100 can automatize the changes according to the stored time patterns, e.g. after a calculated average duration of positioning the patient, infusion changes would occur automatically without manual input by user X. Similar patterns can be applied for ‘asking’ the user to confirm anticipated time frames rather than rely on manual user input.

### Patient-Related Algorithm Adaptation

The automatic anesthesia control system 100 implements correction factors according to patient data, e.g. weight, ASA classification, age, etc., which may be provided through the startup screen 140 of FIG. 4 (see column 2). In an alternative embodiment, the automatic anesthesia control system 100 may take into account additional patient data such as data derived from either the patient's chart or preoperative anesthetic assessment which might have an influence on the pharmacokinetic algorithms or choice of drugs. If a patient is prone to allergic reactions, the automatic anesthesia control system 100 automatically chooses anesthetic agents which are known to cause no or very little allergic reactions and avoid anesthetic agents which do cause allergic reactions. For example, cisatracurium (little allergic potential) may be substituted as a muscle relaxant for rocuronium (high risk of allergic reactions).

This additional patient data may be stored into the database 137 and accessed, for example, by entering an ID associated with the patient in the startup screen 140. It is to be understood that other means may be used to enter the patient data, including manual entry by the user through a user interface.

### Hypnosis Monitoring

The effectiveness of the automatic anesthesia control system 100 strongly depends on the reliability of the input variable, the physiological signal, to be controlled. While the ideal variable to measure the effect of hypnotic drugs is unknown, parameters derived from the analysis of the electroencephalogram (EEG) have emerged as objective and reliable measures of depth of hypnosis for closed loop systems. To this end, a BIS is derived from processing the phase and frequency relations of the component frequencies of the EEG. The BIS is a dimensionless number ranging from 0 (isoelectric activity) to 100 (consciousness). A value between 40 and 60 is considered as representing an adequate state of hypnosis.

Depending on a single input signal, a closed-loop control system may be mislead by artifacts that can occur on the EEG signals, which poses an inherent safety risk for the patient. In order to minimize these artifacts, two indicators, signal quality index (SQI) and electromyography (EMG), are used. SQI reflects the percentage of artifact-free EEG data used to derive the BIS over the previous minute. Artifacts contaminating raw EEG and affecting BIS are usually high frequency signals related either to the use of some surgical instruments and/or to EMG activity. By displaying an EMG signal, both sources of artifacts can be observed.

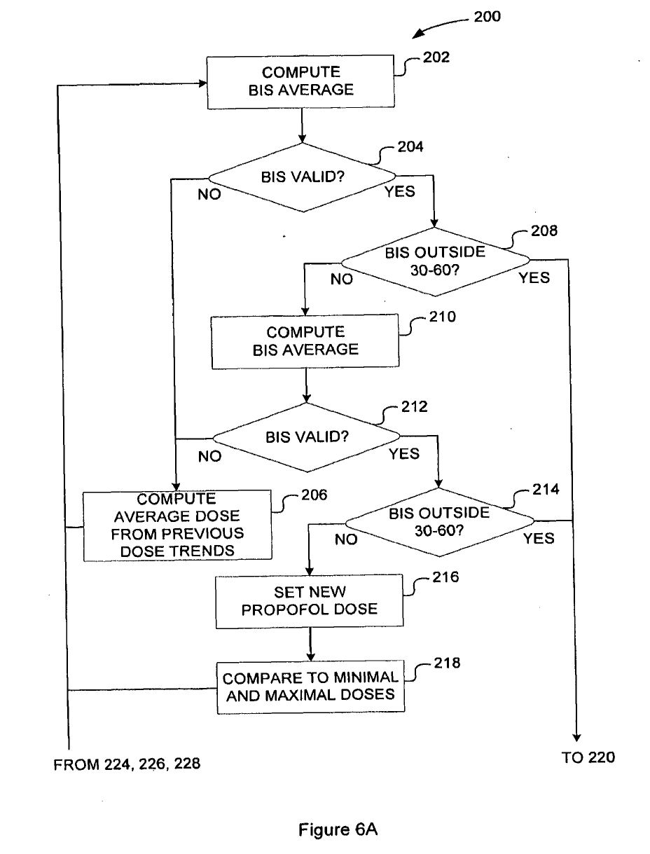

Referring to FIGS. 6A and 6B, there is shown a flow diagram of an illustrative example of the hypnosis monitoring process 200 based on the BIS executed by the processor 131 of the automatic anesthesia control system 100. The steps of the process 200 are indicated by blocks 202 to 228.

The process 200 starts at block 202, where a BIS average is computed for a period of, for example, 15 seconds by applying a moving average to acquired BIS measurements, for example every 5 seconds. Then, at block. 204, the process 200 verifies the validity of the BIS average. A BIS is assumed to be valid when the SQI is greater than 40% and the EMG is lower than 40 dB. If the BIS average is not valid, the process 200 proceeds to block 206 where the drug dose is computed as the average dose from previous dose trends and then returns to block 202.

If the BIS average is valid, the process 200 proceeds to block 208 where it verifies if it is between 30 and 60. If not, it proceeds to block 210; otherwise it proceeds to block 220.

At block 210, a new BIS average is computed and, at block 212, the process 200 verifies if the BIS average is valid. If the BIS average is not valid, the process 200 proceeds to block 206 where the drug dose is computed as the average dose from previous dose trends and then returns to block 202.

If the BIS average is valid, the process 200 proceeds to block 214 where it verifies if it is between 30 and 60. If not, it proceeds to block 216; otherwise it proceeds to block 220.

At block 216, a new drug dose is set based on BIS error, BIS variation and previous BIS trend and, at block 218, is compared to the minimal and maximal doses for the patient. The process 200 then returns to block 202.

At block 220, the process 200 verifies if the BIS average is greater than 60. If not, it proceeds to block 222, if so, it proceeds to block 228 where it injects Bolus based on patient characteristics.

At block 222, the process 200 verifies if the BIS average is lower than 20. If not, it proceeds to block 224, if so, it proceeds to block 226.

At block 224, the process 200 set the infusion to the minimal dose while at block 226, it stops the infusion. In each case the process 200 returns to block 202.

Referring back to FIG. 5, and more specifically to zone 1, BIS values in the range of 40 to 60 indicate a sufficient range of hypnosis. However, in the range of 55-60, there is an inherent risk of too superficial anesthesia and the patient suffering from awareness, a serious condition of intraoperative awakening. Therefore, BIS values in the range of 40 to 55 may be graphically shown in a green area on the BIS scale of 1 to 100 while BIS values in the range of 55 to 60 may be shown in a yellow area. For BIS values from 60 onwards, the state of hypnosis is no longer adequate and immediate action required, hence such BIS values may be shown graphically in a red area. Whereas BIS values below 40 are unnecessarily profound, the patient suffers no harm other than unnecessary profound hypnosis whose clinical impact is debatable; deep hypnosis can lead to low blood pressure and action should be taken, hence such BIS values may be shown graphically in a brown area, but no immediate harm occurs.

It is to be understood that the above described hypnosis monitoring process is provided as an example and that other parameters may be used, for example parameters provided by an auditory evoked potential (AEP) monitor any parameter indicating a degree of unconsciousness.

### Analgesia Monitoring

Pain control during general anesthesia is not easy since the patient cannot talk. However, indirect parameters, such as reactions of the autonomic nerve system, for example sweating, or changes in heart rate or arterial pressure can be used to assess pain. With these parameters, the clinician adjusts analgesia using his judgment, his experience and also surgical variables, such as an estimation of the degree or presence of a surgical stimulus causing pain at any given time during surgery. Out of these parameters, heart rate and blood pressure are the most reliable to assess the pain level during general anesthesia.

Opioids, used during surgery for pain control, are known to effectively block changes in heart rate or blood pressure during periods of surgical stimuli. Although heart rate or blood pressure have been used in surgeries to assess pain there is an absence of studies to establish any kind of ‘intraoperative pain score’ equivalent to the visual pain score widely used to assess pain in the conscious patient.. At present, most studies have solely used either heart rate or blood pressure but not a combination of both to estimate intraoperative pain. In order to translate mean arterial pressure (MAP) or heart rate (HR) variations into possible intraoperative pain, signal processing and interpretation of the data is necessary.

Accordingly, a novel score of pain based on rule based algorithms for mean arterial pressure and heart rate as an objective (albeit indirect) assessment during general anesthesia, the analgoscore, is disclosed in PCT Patent Application publication No. WO 2008/086624. The analgoscore ranges between −9 and 9 indicating either too profound analgesia (−9) or insufficient pain control (9).

Referring back to FIG. 5, and more specifically to zone 2, the analgoscore is shown graphically in colored areas indicating excellent, good or inadequate pain control during general anesthesia. The green area indicates an optimal balance between the dose of analgesia infused and the analgesia provided; the yellow areas still indicate good balance between pain control and analgesic infusion whereas the black areas indicate either insufficient analgesia (9) or too profound analgesia (−9). It should be noted that both areas are colored in black since both they are equally inadequate in clinical practice. The automatic anesthesia control system 100 then uses the information provided by the analgoscore to adjust the level of analgesia drug infusion.

It is to be understood that the above described analgesia monitoring process is provided as an example and that other parameters may be used, for example any parameter indicating a degree of pain.

### Muscle Relaxation Monitoring

There is currently no reliable monitor to determine the complete picture of body muscle relaxation during surgery. In order to properly reflect the relaxation of profound and superficial muscles of the human body, which do not react in the same way, more than one muscle should be reliably monitored. A novel monitoring method disclosed in U.S. Pat. No. 7,236,832, called phonomyography, provides a non-invasive and reliable way of monitoring all muscles of the human body, especially the corrugator supercilii (CS) muscle and the adductor pollicis (AP) muscle.

Two small microphones capable of detecting sound waves of low frequency are used to measure muscle relaxation at the CS muscle and the AP muscle. Muscle contraction is assessed using evoked potentials via two nerve stimulators. Following standard guidelines in anesthesia, the train-of-four ratio (TOF), four electric impulses of 200 msec each at 2 Hz are applied using supramaximal stimulation currents (for the CS muscle: 20-30 mA; for the AP muscle: 40-70 mA), is measured. A TOF-ratio=1 signals normal muscle function while a TOF-ratio<1 signals various degree of muscle relaxation, with TOF-ratios between 0.10 and 0.25 being considered optimal ‘surgical relaxation’.

By monitoring both the AP muscle (representative for peripheral muscle function) and CS muscle (representative for core muscle function, such as abdominal muscles, diaphragm or larynx), the automatic anesthesia control system 100 can adjust the rate of muscle relaxant infusion precisely towards the needed muscle relaxation of the surgical site; e.g. if specifically relaxation of the abdominal cavity is needed like for laparoscopic surgery, the infusion rate will be titrated to maintain a certain level of ‘surgical relaxation’ at the CS muscle as it best reflects the relaxation of the abdominal cavity.

It is to be understood that the above described muscle relaxation monitoring process is provided as an example and that other parameters may be used, for example parameters provided by acceleromyography.

In an alternative embodiment, the automatic anesthesia control system 100 may allow the user to mark the patient as ‘nervous’ through, for example, an added field in the startup screen 140 of FIG. 4 or a dedicated control button 102 or 104, indicating to the automatic anesthesia control system 100 that an increase in the dosing of the hypnotic drug is required, as per common clinical practice.

The induction is then titrated to achieve a given target of hypnosis, analgesia and muscle relaxation and the right moment for intubation indicated to the user through, for example, an added intubation indicator in the maintenance screen 150 of FIG. 5. Only when a BSI of less than 40, an analgoscore within the green area and a muscle relaxation of more than 90% at the core (CS muscle) is achieved, is the intubation indicator activated to notify the user that the conditions for intubation are optimal. It is to be understood that if other control variables are used, the conditions for the activation of the intubation indicator will vary accordingly.

### Use of the Automatic Anesthesia Control System for Sedationj

In an alternative embodiment, the automatic anesthesia control system 100 may also be provided with the ability to automatically control the sedation of a patient. To this end, the automatic anesthesia control system 100 may be provided with appropriate additional user interfaces. In a further alternative embodiment, a “lighter” version of the automatic anesthesia control system 100 may be provided which only controls the sedation of a patient.

Sedation during surgery is an important anesthetic task; especially in orthopedic surgery, patients undergo procedures in spinal anesthesia and sedation using, for example, propofol.

This is the standard practice in patients undergoing hip or knee replacement surgery. Most anaesthesiologists use clinical judgment and an objective monitoring parameter, e.g. the BIS provided by Aspect Medical Systems, to titrate the continuous infusion of propofol during these procedures.

Using hypnosis monitoring, the infusion rate of propofol is then adjusted by the automatic anesthesia control system 100 to the patient's needs whilst maintaining his or her spontaneous breathing activity.

Sedation can also be used during non-surgical interventions (e.g. endoscopic examinations of the upper or lower gastro-intestinal tract, cardioavascalar examinations, such as transesophageal echocardiography or any other similar intervention).

This type of sedation is very often performed without the immediate presence of an anaesthesiologist. Propofol is in these instances very often administered by non-trained personnel with the inherent risk of under-or overdosing resulting in severe cardiovascular or respiratory complications.

The risks of sedation during surgery or these interventions are either over-sedation with propofol, which can cause hypotension or reduced spontaneous breathing activity, accumulation of CO₂ and hypoxia or under-sedation with the inherent risk of spontaneous movements, which can endanger the success of surgery, or patient discomfort. Almost all patients prefer to be ‘asleep’ during these procedures despite being rendered completely pain-free by the spinal anesthesia. Dosing problems can result in hemodynamic compromise and inherent risk for the life and well-being of the patient. The automatic anesthesia control system 100 can be used to help avoid risks by detecting critical limits earlier and alerting the user using pop-up menus.

Accordingly, the automatic anesthesia control system 100 can be used to maintain any level of sedation. As monitoring parameter, any discrete consciousness monitor, such as the BIS, can be used.

In addition, the automatic anesthesia control system 100 integrates the following vital sign parameters: peripheral oxygen saturation, blood pressure and heart rate, breathing rate, expiratory carbon dioxide and the shape of the expiratory carbon dioxide curve.

The automatic anesthesia control system 100 also integrates the amount of oxygen flow to the patient as well as movement via, for example, phonomyographic sensors applied to the body of the patient 1. From these, the automatic anesthesia control system 100 is able to detect movement of the patient's body as well as the complete hemodynamic and respiratory status.

The automatic anesthesia control system 100 then uses the information provided by the vital sign parameters, oxygen flow and movement to adjust the level of drug infusion.

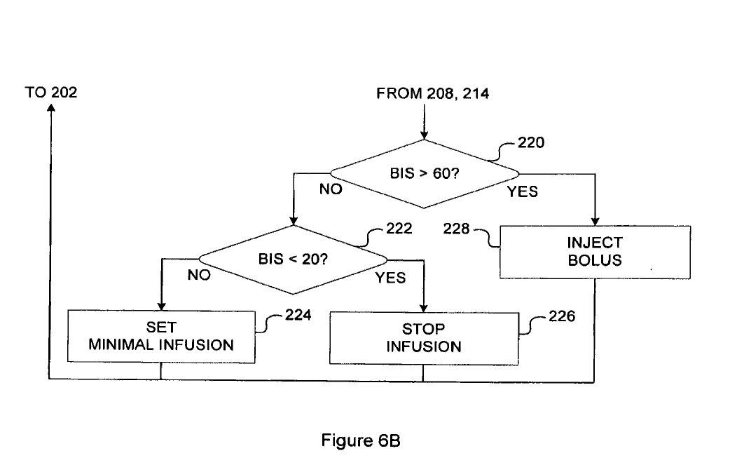

Referring to FIG. 7, these parameters may be integrated into a sedation user screen 170 which is a simplified version of the startup screen 140 of FIG. 4 and the maintenance screen 150 of FIG. 5, the sedation user interface 170 displaying the elapsed time, patient information, vital signs of the patient, BIS (or any other hypnosis monitoring parameter), propofol dosage (or any other drug), saturation, respiratory rate and graphical display of the BIS trend.

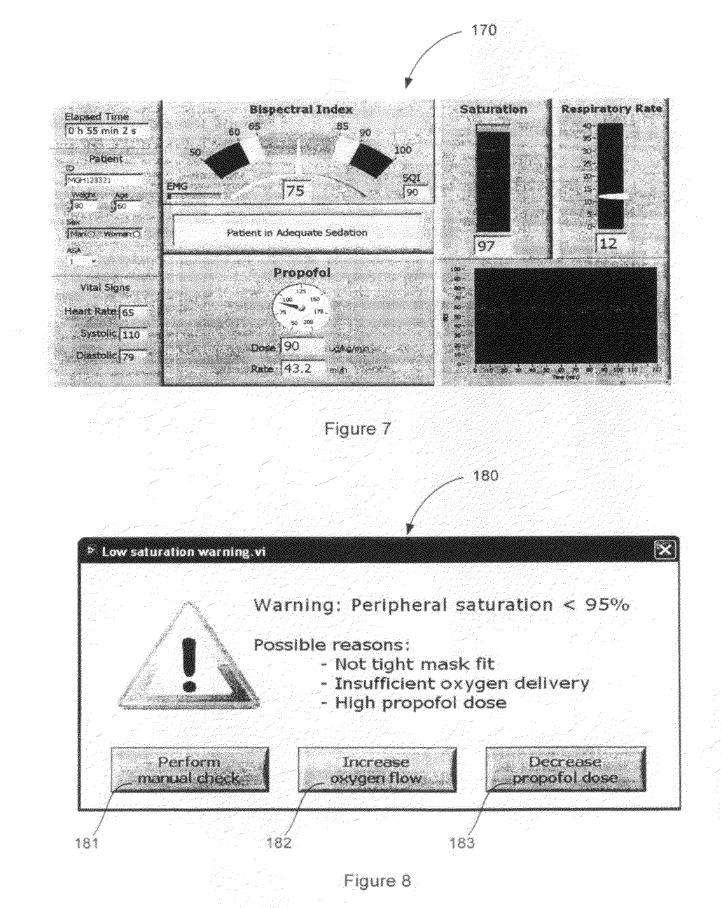

When used as a decision assist system, the automatic anesthesia control system 100 can display pop-alerts of different kinds which help the user to perform certain tasks. For example, FIG. 8 shows a low saturation warning pop-alert 180 alerting the user of a low peripheral saturation and allowing the user to, by pressing an appropriate button, perform a manual check 181, increase the oxygen flow 182 or decrease the propofol does (or any other drug).

Wireless Communication with a Handheld System

In an alternative embodiment, the automatic anesthesia control system 100 may communicate with handheld devices such as a personal digital assistant using, for example, wireless communication port 116. The display 106 may then be remotely displayed using the handheld device which may be used to communicate either unidirectionally (as an information tool only) or bi-directionally (allowing the modification of operating parameters) with the automatic anesthesia control system 100.

It is to be understood that, further to the user interfaces disclosed above, various alarms and conditions status may be generated such as, for example, “NOT INFUSING” when the infusion is stopped, “NEARLY EMPTY” when a syringe 112 needs to be replaced. Alarms can alert the user about critical events such as the injection of automatic bolus, the injection of neuromuscular blocker, the presence of artifacts in the BIS signal, the accidental loss of connectivity with any device (monitor, pumps etc).

Although the present invention has been described by way of particular embodiments and examples thereof, it should be noted that it will be apparent to persons skilled in the art that modifications may be applied to the present particular embodiment without departing from the scope of the present invention.

What is claimed is:

1. An anesthesia control system for controlling at least one component of anesthesia of a patient, comprising:
an input for acquiring a target parameter indicative of a current degree of the at least one component of anesthesia of the patient from a monitoring module;
an output for connecting to a drug delivery mechanism being so configured so as to be intravenously infuse a drug to the patient;
a user interface for activating and controlling the control system;
a processor operatively connected to the input, the output and the user interface, the processor being so configured so as to:
calculate the necessary dose of a drug for achieving a desired degree of the at least one component of anesthesia based on the target parameter; and
providing the calculated drug dose to the output.

2. A system in accordance with claim 1, wherein the processor is further configured so as to display through the user interface the current degree of the at least one component of anesthesia and the calculated drug dose.

3. A system in accordance with claim 1, wherein the drug delivery mechanism is included within the anesthesia control system.

4. A system in accordance with claim 3, wherein the drug delivery mechanism is selected from a group consisting of at least one syringe drive for loading a syringe containing the drug with and a syringe drive with a drug cartridge system for loading a cartridge containing the drug.

5. A system in accordance with claim 4, wherein drug cartridge system includes an automatic drug recognition system for preventing false drug application.

6. A system in accordance with claim 5, wherein the automatic drug recognition system is based on a bar code recognition system.

7. A system in accordance with claim 1, wherein the monitoring module is included within the anesthesia control system.

8. A system in accordance with claim 1, further comprising a database operatively connected to the processor.

9. A system in accordance with claim 8, wherein a user can select a type of surgery using the user interface and wherein the processor is further configured so as to adjust the calculated necessary dose of the drug based on adjustment patterns associated with the selected type of surgery stored in the database.

10. A system in accordance with claim 8, wherein the processor is further configured so as to adjust the calculated necessary dose of the drug based on learned patterns from pass surgeries of the same type stored in the database.

11. A system in accordance with claim 8, wherein the processor is further configured so as to select the drug based on allergies of the patient stored in the database.

12. A system in accordance with claim 1, wherein the at least one component of anesthesia is selected from a group consisting of hypnosis, analgesia and muscle relaxation.

13. A system in accordance with claim 1, wherein the at least one component of anesthesia includes hypnosis and wherein the target parameter is an estimate of the degree of unconsciousness of the patient.

14. A system in accordance with claim 13, wherein the calculation of the dose is further based on at least one of vital signs, oxygen flow and movement of the patient.

15. A system in accordance with claim 1, wherein the at least one component of anesthesia includes hypnosis and wherein the target parameter is a function of the phase and frequency of an electroencephalogram of the patient.

16. A system in accordance with claim 15, wherein the target parameter is a bispectral index.

17. A system in accordance with claim 16, wherein the calculation of the dose is further based on a signal quality index of the electroencephalogram and an electromyography of the patient.

18. A system in accordance with claim 1, wherein the at least one component of anesthesia includes analgesia and wherein the target parameter is an estimate of the degree of pain of the patient.

19. A system in accordance with claim 1, wherein the at least one component of anesthesia includes analgesia and wherein the target parameter is a function of the arterial pressure and heart rate of the patient.

20. A system in accordance with claim 19, wherein the target parameter is an analgoscore.

21. A system in accordance with claim 1, wherein the at least one component of anesthesia includes muscle relaxation and wherein the target parameter is a function of the evoked potential of at least one target muscle.

22. A system in accordance with claim 21, wherein the at least one target muscle includes the corrugator supercilii and adductor pollicis muscles.

23. A system in accordance with claim 21, wherein the target parameter is a phonomyography.

24. A method for controlling at least one component of anesthesia of a patient, comprising the step of:
acquiring a target parameter indicative of a current degree of the at least one component of anesthesia;
calculating the necessary dose of a drug for achieving a desired degree of the at least one component of anesthesia based on the target parameter; and
delivering the drug to the patient via an infusion drip.

25. A method in accordance with claim 24, wherein the at least one component of anesthesia is selected from a group consisting of hypnosis, analgesia and muscle relaxation.

26. A method in accordance with claim 24, wherein the at least one component of anesthesia includes hypnosis and wherein the target parameter is an estimate of the degree of unconsciousness of the patient.

27. A method in accordance with claim 26, wherein the calculation of the dose is further based on at least one of vital signs, oxygen flow and movement of the patient.

28. A method in accordance with claim 24, wherein the at least one component of anesthesia includes hypnosis and wherein the target parameter is a function of the phase and frequency of an electroencephalogram of the patient.

29. A method in accordance with claim 27, wherein the at least one component of anesthesia includes hypnosis and wherein the target parameter is a bispectral index.

30. A method in accordance with claim 29, wherein the calculation of the dose is further based on a signal quality index of the electroencephalogram and an electromyography of the patient.

31. A method in accordance with claim 24, wherein the at least one component of anesthesia includes analgesia and wherein the target parameter is an estimate of the degree of pain of the patient.

32. A method in accordance with claim 24, wherein the at least one component of anesthesia includes analgesia and wherein the target parameter is a function of the arterial pressure and heart rate of the patient.

33. A method in accordance with claim 32, wherein the at least one component of anesthesia includes analgesia and wherein the target parameter is an analgoscore.

34. A method in accordance with claim 24, wherein the at least one component of anesthesia includes muscle relaxation and wherein the target parameter is a function of the evoked potential of at least one target muscle.

35. A method in accordance with claim 34, wherein the at least one target muscle includes the corrugator supercilii and adductor pollicis muscles.

36. A method in accordance with claim 24, wherein the at least one component of anesthesia includes muscle relaxation and wherein the target parameter is a phonomyography.

37. A method in accordance with claim 24, wherein the step of calculating the necessary dose of the drug is further based on the type of surgery.

38. A method in accordance with claim 24, wherein the step of calculating the necessary dose of the drug is further based on learned patterns from pass surgeries of the same type.

39. A method in accordance with claim 24, further comprising the step of selecting the drug based on allergies of the patient.
